(19) United States
(12) Patent Application Publication
(10) Pub. No.: US 2008/0070266 A1
(43) Pub. Date: Mar. 20, 2008

(54) FRET-based apoptosis detector

(75) Inventors: Andrzej Myc, Ann Arbor, MI (US); Istvan J. Majoros, Ann Arbor, MI (US); Thommey P. Thomas, Dexter, MI (US); James R. Baker JR., Ann Arbor, MI (US)

Correspondence Address: Casimir Jones, S.C., 440 Science Drive, Suite 203, Madison, WI, 53711, US

(73) Assignee: Regents of the University of Michigan, Ann Arbor, MI (US)

(21) Appl. No.: 11/827,232

(22) Filed: Jul. 11, 2007

Related U.S. Application Data
(60) Provisional application No. 60/819,998, filed on Jul. 11, 2006.

(51) Int. Cl. C12Q 1/37; C12Q 1/02

(52) U.S. Cl. 435/24; 435/29

(57) ABSTRACT

The present invention relates to compositions comprising a FRET-based substrate, a cell-targeting moiety and a dendrimer, and methods for generating and using the same.

The present application claims priority to U.S. Provisional Patent Application Ser. No. 60/819,998, filed Jul. 11, 2006, which is herein incorporated by reference in its entirety.

The present invention was made, in part, with government support under NIH-NCI Contract No. No1-CM-97065-32. The government may have certain rights in the invention.

### FIELD OF THE INVENTION

The present invention relates to compositions comprising a FRET-based substrate, a cell-targeting moiety and a dendrimer, and methods for generating and using the same.

### BACKGROUND

Apoptosis is an important process in maintaining tissue homeostasis, controlling abnormal cell growth and regulating the immune system. Proteolytic enzymes called caspases play a key role in apopotosis. Activation of caspase-3, one of the cysteine proteases, is a hallmark of apoptosis. Caspase-3 has a high specificity to cleave proteins that contain the sequence valine-aspartic acid. However, commercially available caspase-3 substrates suitable for detection based on the principle of fluorescence resonance energy transfer (FRET) are nonspecific and target all cell types. Thus, there is a need for enzyme specific apoptosis detection methods that are narrowly able to target cell types of interest, for example, neoplastic cells.

### SUMMARY OF THE INVENTION

The present invention relates to compositions comprising a FRET-based substrate, a cell-targeting moiety and a dendrimer, and methods for generating and using the same.

Accordingly, in some embodiments, the present invention provides a composition comprising a FRET-based substrate, a cell targeting moiety and a dendrimer. In some embodiments of the present invention the FRET-based substrate is PhiPhiLux™ G₁D₂. The present invention is not limited by the type of FRET-based substrate. Indeed, a variety of FRET-based substrates are contemplated to be useful in the present invention. In other embodiments, the cell-targeting moiety includes, but is not limited to, an antibody, a receptor ligand, a hormone, a vitamin, and an antigen, however, the present invention is not limited by the nature of the targeting agent. In some embodiments, the antibody is specific for a disease-specific antigen. In further embodiments, the disease-specific antigen comprises a tumor-specific antigen. In still further embodiments, the receptor ligand includes, but is not limited to, a ligand for CFTR, EGFR, the estrogen receptor, FGR2, folate receptor, IL-2 receptor, glycoprotein, and VEGFR. In a preferred embodiment, the receptor ligand-cell-targeting moiety is folic acid. Other embodiments that may be used with the present invention are described in U.S. Pat. No. 6,471,968 and WO 01/87348, each of which is herein incorporated by reference in their entireties. In particularly preferred embodiments, the dendrimer is PAMAM G5. The present invention is not limited by the type of dendrimer. Indeed, a variety of dendrimers are contemplated to be useful in the present invention.

In some embodiments, the present invention provides a method to detect apoptosis comprising providing a cell, a nanodevice comprising a FRET-based substrate, a cell-targeting moiety and a dendrimer, wherein the FRET-based substrate, the cell-targeting moiety and the dendrimer comprise a stable conjugate, and contacting the cell with the nanodevice and detecting a change in the level of an intracellular fluorescent signal indicating the presence or absence of apoptosis of the cell. In some embodiments of the present invention apoptosis is caspase-3 mediated apoptosis. In other embodiments, the FRET-based substrate is PhiPhiLux™ G₁D₂. The present invention is not limited by the type of FRET-based substrate. Indeed, a variety of FRET-based substrates are contemplated to be useful in the present invention. In further embodiments, the cell-targeting moiety includes, but is not limited to, an antibody, a receptor ligand, a hormone, a vitamin, and an antigen, however, the present invention is not limited by the nature of the targeting agent. In some embodiments, the antibody is specific for a disease-specific antigen. In further embodiments, the disease-specific antigen comprises a tumor-specific antigen. In still further embodiments, the receptor ligand includes, but is not limited to, a ligand for CFTR, EGFR, the estrogen receptor, FGR2, folate receptor, IL-2 receptor, glycoprotein, and VEGFR. In a preferred embodiment, the receptor ligand-cell-targeting moiety is folic acid. Other embodiments that may be used with the present invention are described in U.S. Pat. No. 6,471,968 and WO 01/87348, each of which is herein incorporated by reference in their entireties. In some embodiments, the dendrimer is PAMAM G5. The present invention is not limited by the type of dendrimer. Indeed, a variety of dendrimers are contemplated to be useful in the present invention.

In some embodiments of the present invention the cell is folate receptor α positive. In other embodiments the cell is a neoplastic cell. In further embodiments, FRET-based detection is by flow cytometry. In some embodiments, the method of detection is in vitro. In other embodiments the method of detection is in vivo.

In some embodiments, the present invention provides a kit comprising reagents useful for, or sufficient for, carrying out a method of the present invention.

In some embodiments the present invention provides a method of synthesizing a FRET-based apoptsis detecting nanodevice comprising providing a FRET-based substrate, a cell targeting moiety and a dendrimer. In some embodiments of the present invention the FRET-based substrate is PhiPhiLux™ G₁D₂. The present invention is not limited by the type of FRET-based substrate. Indeed, a variety of FRET-based substrates are contemplated to be useful in the present invention. In other embodiments, the cell-targeting moiety includes, but is not limited to, an antibody, a receptor ligand, a hormone, a vitamin, and an antigen, however, the present invention is not limited by the nature of the targeting agent. In some embodiments, the antibody is specific for a disease-specific antigen. In further embodiments, the disease-specific antigen comprises a tumor-specific antigen. In still further embodiments, the receptor ligand includes, but is not limited to, a ligand for CFTR, EGFR, the estrogen receptor, FGR2, folate receptor, IL-2 receptor, glycoprotein, and VEGFR. In a preferred embodiment, the receptor ligand-cell-targeting moiety is folic acid. Other embodiments that may be used with the present invention are described in U.S. Pat. No. 6,471,968 and WO 01/87348, each of which is herein incorporated by reference in their entireties. In particularly preferred embodiments, the dendrimer is PAMAM G5. The present invention is not limited by the type of dendrimer. Indeed, a variety of dendrimers are contemplated to be useful in the present invention. In some embodiments, the method of synthesizing the FRET-based apoptosis detection composition of the present invention is by partial acetylation of the dendrimer, conjugation of folic acid to the partially acetylated dendrimer via condensation, conjugation of the FRET-based substrate via reaction of the FRET-based substrate in a solvent mixture of DMF:DMSO followed by addition of the FRET-based substrate to the partially acetylated dendrimer folic acid conjugate, and subsequent filtration and lyophilization. In some embodiments of the present invention, the functional group is attached to the dendrimer by a linker molecule. In other embodiments, the functional group is directly attached to the dendrimer. The present invention is not limited by the order in which the functional groups and groups are added to the dendrimer.

In some embodiments, the present invention provides a method of monitoring treatment for a disease comprising administering the FRET-based apoptosis detection composition of the present invention to a subject suffering from, or susceptible to, a disease, and detecting the amount of apoptosis in a cell from said subject after a medical or surgical treatment. In some embodiments, the detection is in vivo detection, for example, via direct observation or non-invasive imaging. In other embodiments, the detection is in vitro detection, for example, via direct observation or imaging of a sample from a subject.

### BRIEF DESCRIPTION OF THE DRAWINGS

FIG. 1 depicts a synthesis scheme of a bi-functional PAMAM dendritic device in one embodiment of the present invention. The synthetic scheme for order of syntheses is: 1. G5 carrier; 2. G5-Ac(96); 3. G5-Ac(96)-FA; 4. G5-Ac(96)-FA-PhiPhiLux™ G₁D₂.

FIG. 2 shows the GPC RI and light scattering signal (90°) of the G5 dendrimer (FIG. 2A) and the G5-Ac(96 (FIG. 2B) partially acetylated dendrimer.

FIG. 3 shows the ¹H NMR of the G5-Ac(96)-FA(5) conjugate.

FIG. 4 shows the HPLC eluogram of the G5-Ac(96)-FA(5) conjugate (1) before (FIG. 4A) and (2) after (FIG. 4B) membrane filtration purification.

FIG. 5 shows a PhiPhiLux™ G₁D₂ structure with (1) a carboxyl group participating in the conjugation, and (2) cleavage by caspase-3 enzyme.

FIG. 6 shows the fluorescent intensity of Jurkat cells stained with PhiPhiLux™ G₁D₂. FIG. 6A shows the background fluorescence of unstained cells. FIG. 6B shows fluorescence of control stained cells. FIG. 6C shows fluorescence of apoptotic stained cells.

FIG. 7A shows the fluorescent intensity of KB cells, and FIG. 7B shows the fluorescent intensity of UMSCC-38 cells stained with a G5-Ac(96)-FA-PhiPhiLux™ G₁D₂ nanodevice.

### DEFINITIONS

To facilitate an understanding of the present invention, a number of terms and phrases are defined below:

As used herein, the term “agent” refers to a composition that possesses a biologically relevant activity or property. Biologically relevant activities are activities associated with biological reactions or events, or that allow the detection, monitoring, or characterization of biological reactions or events. Biologically relevant activities include, but are not limited to, therapeutic activities (e.g., the ability to improve biological health or prevent the continued degeneration associated with an undesired biological condition), targeting activities (e.g., the ability to bind or associate with a biological molecule or complex), monitoring activities (e.g., the ability to monitor the progress of a biological event or to monitor changes in a biological composition), imaging activities (e.g., the ability to observe or otherwise detect biological compositions or reactions), and signature identifying activities (e.g., the ability to recognize certain cellular compositions or conditions and produce a detectable response indicative of the presence of the composition or condition). The agents of the present invention are not limited to these particular illustrative examples. Indeed any useful agent may be used including agents that deliver or destroy biological materials, cosmetic agents, and the like. In preferred embodiments of the present invention, the agent or agents are associated with at least one dendrimer (e.g., incorporated into the dendrimer, surface exposed on the dendrimer, etc.). In some embodiments of the present invention, one dendrimer is associated with two or more agents that are “different than” each other (e.g., one dendrimer associated with both a targeting agent and a therapeutic agent). “Different than” refers to agents that are distinct from one another in chemical makeup and/or functionality.

As used herein, the terms “functionalized” refer generally to a dendrimer wherein charge reducing molecules have been substituted for terminal amine groups present within the dendrimer. The present invention is not limited to acetamide and hydroxyl groups. Indeed, any charge reducing molecule that can be substituted for terminal amine groups and that reduces the overall net charge of the dendrimer use in the present invention.

As used herein, the term “nanodevice” refers to small (e.g., invisible to the unaided human eye) compositions containing or associated with one or more “agents.” In its simplest form, the nanodevice consists of a physical composition (e.g., a dendrimer, a dendrimer encapsulated nanoparticle, or a dendrite) associated with at least one agent that provides biological functionality (e.g., a therapeutic agent or a diagnostic agent). However, the nanodevice may comprise additional components (e.g., additional dendrimers and/or agents).

The term “biologically active,” as used herein, refers to a protein or other biologically active molecules (e.g., catalytic RNA or small molecule) having structural, regulatory, or biochemical functions of a naturally occurring molecule.

The term “agonist,” as used herein, refers to a molecule which, when interacting with a biologically active molecule, causes a change (e.g., enhancement) in the biologically active molecule, which modulates the activity of the biologically active molecule. Agonists may include proteins, nucleic acids, carbohydrates, or any other molecules that bind or interact with biologically active molecules. For example, agonists can alter the activity of gene transcription by interacting with RNA polymerase directly or through a transcription factor.

The terms “antagonist” or “inhibitor,” as used herein, refer to a molecule which, when interacting with a biologically active molecule, blocks or modulates the biological activity of the biologically active molecule. Antagonists and inhibitors may include proteins, nucleic acids, carbohydrates, or any other molecules that bind or interact with biologically active molecules. Inhibitors and antagonists can affect the biology of entire cells, organs, or organisms (e.g., an inhibitor that slows tumor growth).

The term “modulate,” as used herein, refers to a change in the biological activity of a biologically active molecule. Modulation can be an increase or a decrease in activity, a change in binding characteristics, or any other change in the biological, functional, or immunological properties of biologically active molecules.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ non-translated sequences. The sequences that are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

The term “antigenic determinant” as used herein refers to that portion of an antigen that makes contact with a particular antibody (e.g., an epitope). When a protein or fragment of a protein is used to immunize a host animal, numerous regions of the protein may induce the production of antibodies which bind specifically to a given region or three-dimensional structure on the protein; these regions or structures are referred to as antigenic determinants. An antigenic determinant may compete with the intact antigen (e.g., the “immunogen” used to elicit the immune response) for binding to an antibody.

The terms “specific binding” or “specifically binding” when used in reference to the interaction of an antibody and a protein or peptide means that the interaction is dependent upon the presence of a particular structure (e.g., the antigenic determinant or epitope) on the protein; in other words the antibody is recognizing and binding to a specific protein structure rather than to proteins in general. For example, if an antibody is specific for epitope “A,” the presence of a protein containing epitope A (or free, unlabelled A) in a reaction containing labeled “A” and the antibody will reduce the amount of labeled A bound to the antibody.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The term “test compound” refers to any chemical entity, pharmaceutical, drug, and the like that can be used to treat or prevent a disease, illness, sickness, or disorder of bodily function. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables).

As used herein, the term “dendrimers” refers to nearly spherical, highly branched macromolecules with symmetrically emanating dendrons of defined molecular weight and size. As used herein, the term “dendrites” refers to macromolecules with a main branch or trunk, from which grow side branches, from which grow smaller side branches, and so on.

As used herein, the term “apoptosis” refers to a process in which a cell actively participates in its own destruction.

As used herein, the term “FRET-based” substrate refers to any molecule comprising a fluorophore/quencher system.

As used herein, the term “cell targeting moiety” refers to a molecule that provides a specific interaction with a cell type vs. other cell types. In some embodiments of the present invention, the “cell targeting moiety” interacts with one type of cell, for example a neoplastic cell, and substantially not with other types of cells, for example non-neoplastic cells.

As used herein, the term “stable conjugate” refers to a covalent or non-covalent complex that remains affixed under reaction conditions including, for example, delivery of the complex to a cell.

As used herein, the term “neoplastic cell” refers to a cell in a tumor, an abnormal growth of tissue, or a neoplasm.

### DESCRIPTION OF THE INVENTION

The present invention relates to compositions comprising a FRET-based substrate, a cell-targeting moiety and a dendrimer, and methods for generating and using the same. Compositions comprising the nanodevices of the present invention find use in a variety of settings including, but not limited to, therapeutic, diagnostic and research applications.

Apoptosis, or programmed cell death (PCD), is an important process in maintaining tissue homeostasis, controlling abnormal cell growth and regulating the immune system. (Zou, C.-P., Youssed, E. M., Zou, C.-C., Carey, T. E., and Lotan, R. (2001). Differential effects of chromosome 3p deletion on the expression of the putative tumor suppressor rarβ and on retinoid resistance in human squamous carcinoma cells. Oncogene 20, 6820-6827, Okada, H., and Mak, T. W. (2004). Pathways of apoptotic and non-apoptotic death in tumour cells. Nature Cancer Reviews 4, 592-603, Arrends M. J., and Wylie A. H. (1991). Apoptosis: Mechanisms and roles in pathology. Int. Rev. Exp. Pathol. 32, 223-254, Thompson C. B. (1995). Apoptosis in the pathogenesis and treatment of disease. Science 267, 1456-1462, Rudin, C. M., and Thompson, C. B. (1997). Apoptosis and disease: regulation and clinical relevance of programmed cell death. Annual Review of Medicine 48, 267-281). The term apoptosis is used to describe a process in which a cell actively participates in its own destruction. The duration of this destructive process differs by cell type and can be influenced by the presence of inducing or inhibiting agents. Specific morphological, biochemical and molecular changes characterize the apoptotic cascade. PCD leads to characteristic cell morphological changes that include cell fragmentation, chromatin condensation, membrane blebbing, and cytoplasmic shrinkage. (Wyllie, A. H., Kerr, J. F., and Currie, A. R. (1980). Cell death: the significance of apoptosis. International Review of Cytology 68, 251-306). The central component of PCD is a cascade of proteolytic enzymes called caspases, a structurally related group of cysteine aspartate-specific proteases. (Slee, E. A., Adrian, C., and Martin, S. J. (1999). Serial killers: Ordering caspase activation events in apoptosis. Cell Death and Differ. 6, 1067-1074). Caspase-3 is one of the cysteine proteases most frequently activated during the process of apoptosis. Activation of the caspase family is one of the earliest markers of an apoptotic event. While apoptosis is possibly reversible if detected in its earliest stages, once caspase activity has begun, the process becomes irreversible. The final phases of apoptosis require the activation of the caspase family.

In response to pro-apoptotic stimuli, the 32 kDa pro-Caspase-3 is processed to an active enzyme consisting of two subunits of 17 and 12 kDa. Activated caspase-3 is essential for the progression of apoptosis, resulting in the degradation of cellular proteins, apoptotic chromatin condensation, and DNA fragmentation; it also has a high specificity to cleave proteins that contain the sequence valine-aspartic acid. (Wyllie, A. H. (1980). Glucocorticoid-induced thymocyte apoptosis is associated with endogenous endonuclease activation. Nature 284, 555-556.) Based on this principle, several fluorogenic substrates have been developed to detect active caspase-3 in cells. (Pozarowski, P., Huang, X., Halicka, D. H., Lee, B., Johnson, G., and Darzynkiewicz, Z. (2003). Interactions of fluorochrome-labeled caspase inhibitors with apoptotic cells: A caution in data interpretation. Cytometry 55A, 50-60, Belloc, F., Belaud-Rotureau, M. A., Lavignolle, V., Bascans, E., Braz-Pereira, E., Durrieu, F., and Lacombe, F. (2000). Flow cytometry detection of caspase 3 activation in preapoptotic leukemic cells. Cytometry 40, 151-160).

In the absence of active capase-3, these substrates remain non-fluorescent due to fluorescence resonance energy transfer (FRET) between donor and acceptor subunits on the oligopeptide. (Pozarowski, P., Huang, X., Halicka, D. H., Lee, B., Johnson, G., and Darzynkiewicz, Z. (2003). Interactions of fluorochrome-labeled caspase inhibitors with apoptotic cells: A caution in data interpretation. Cytometry 55A, 50-60). In apoptotic cells, active caspase-3 cleaves the oligopeptide between valine-aspartic acid, releasing the fluorescent module. The amount of fluorescence can then be quantified by flow cytometry. ((Pozarowski, P., Huang, X., Halicka, D. H., Lee, B., Johnson, G., and Darzynkiewicz, Z. (2003). Interactions of fluorochrome-labeled caspase inhibitors with apoptotic cells: A caution in data interpretation. Cytometry 55A, 50-60, Belloc, F., Belaud-Rotureau, M. A., Lavignolle, V., Bascans, E., Braz-Pereira, E., Durrieu, F., and Lacombe, F. (2000). Flow cytometry detection of caspase 3 activation in preapoptotic leukemic cells. Cytometry 40, 151-160). These types of substrates may be of value when monitoring apoptosis in vitro and in vivo. There is also a great need for a highly specific apoptosis detection device that detects apoptosis only in certain types of cells or tissues. A highly specific detection device is especially needed when attempting to detect apoptosis in organs and tissues in vivo. To the present, all commercially available FRET-based fluorogenic substrates are nonspecific and target nearly all cell types.

In the course of experiments leading to the development of the present invention it was determined which of the commercially available substrates yields the highest discrimination between apoptotic and non-apoptotic cells. Next, this substrate was conjugated to a G5 dendrimer designed specifically to detect apoptosis. These experiments led to the synthesis and testing of an apoptosis detector nanodevice that specifically targets cells via the folate receptor, a protein that is over-expressed in many types of cancers. The non-specific FRET-based apoptosis detector PhiPhiLux™ G₁D₂ is commercially available from Calbiochem (San Diego, Calif.). For a carrier, the PAMAM dendrimer generation 5 (G5) was selected. Both the PhiPhiLux™ G₁D₂ (apoptosis detector) and folic acid (FA) were then successively conjugated to the G5 dendrimer in experiments conducted in the course of development of the present invention. The nanodevice of the present invention preferably combines two functions: 1.) intracellular targeting; and 2) detecting apoptosis. For example, G5-Ac(96)-FA-PhiPhiLux™ G₁D₂, and detects apoptosis specifically in KB cells overexpressing the high affinity folate receptors a.

The experimental examples below show that the G5-Ac(96)-FA-PhiPhiLux™ G₁D₂ nanodevice specifically detects apoptosis in targeted cells (e.g., KB cells). The G5-Ac(96)-FA-PhiPhiLux™ G₁D₂ nanodevice is the first targeted apopotosis detector that has been developed and is useful, for example, to monitor the response to therapy in cells receiving cancer chemotherapeutics in vitro and in vivo.

The following discussion describes individual component parts of the dendrimer and methods of making and using the same in some embodiments of the present invention. To illustrate the design and use of the methods and compositions of the present invention, the discussion focuses on specific embodiments of the use of the compositions, for example, in the monitoring of breast adenocarcinoma and colon adenocarcinoma. These specific embodiments are intended only to illustrate certain preferred embodiments of the present invention and are not intended to limit the scope thereof (e.g., compositions and methods of the present invention find use in the identification of prostate cancer and virally infected cells and tissue). In some embodiments, the FRET-based apoptosis nanodevices of the present invention target neoplastic cells through cell-surface moieties and are taken up by the tumor cell for example through receptor mediated endocytosis. As is clear from the examples below, the use of the compositions of the present invention facilitate non-intrusive sensing, signaling, monitoring and diagnosis for cancer and other diseases and conditions.

Dendrimers

In the present invention, dendrimers (e.g., polyamidoamine (PAMAM) dendrimers) serve as templates or stabilizers (See, e.g., Balogh and Tomalia, J. Am. Chem. Soc. 1998, 120, 7355-7356; Esumi et al., Langmuir 1998, 14, 3157-3159; Crooks et al., Accounts Chem. Res. 2001, 34, 181-190; Zhao et al., J. Am. Chem. Soc. 1998, 120, 4877-4878). Dendrimers (e.g., PAMAM dendrimers) are close to spherical, highly branched macromolecules with symmetrically emanating dendrons of defined molecular weight and size (See, e.g., U.S. Pat. No. 6,471,968, and U.S. Pat. App. Nos. 60/604,321, filed Aug. 25, 2004, and 60/690,652, filed Jun. 15, 2005, herein incorporated by reference in their entireties). Dendrimers of the present invention are composed of a core molecule and dendritic branches that regularly extend from the core to terminal groups (See, Tomalia et al., Polymer J. 1985, 17, 117; Tomalia et al., Macromolecules 1986, 19,2466-2468; Tomalia et al., Angew. Chem. Int. Ed. Engl. 1990, 29, 138). Dendrimers (e.g., PAMAMs) have a narrow polydispersity and are ideal stabilizers to encapsulate and stabilize metal nanoparticles due to their “built-in” functional groups, fairly uniform composition and defined structures. Organic/inorganic hybrid metal dendrimer NPs hold great promise in various applications such as catalysis (See, e.g., Zhao and Crooks, Angew. Chem. Int. Ed. 1999, 38, 364-366), optics (See, e.g., Ispasoiu et al., J. Am. Chem. Soc. 2000, 122, 11005-11006; Ye et al., Appl. Phys. Lett. 2002, 80, 1713-1715), biological sensing (See, e.g., Bielinska et al., J. Nanoparticle Res. 2002, 4, 395-403), cancer therapeutics (See, e.g., Balogh et al., Chimica Oggi/Chemistry Today 2002, 20, 35-40), and building blocks to assemble functional films (See, e.g., He et al., Chem. Mater. 1999, 11, 3268-3274; Esumi et al., Langmuir 2003, 19, 7679-7681).

Although an understanding of the mechanism is not necessary to practice the present invention, and the present invention is not limited to any particular mechanism of action, in some embodiments, it is expected that decreasing the surface charge of amine-terminated PAMAM dendrimers (e.g., towards or to neutral) reduces their in vivo toxicity. For example, in some embodiments, decreasing (e.g., neutralizing) the surface charge of amino-terminated PAMAM dendrimers is achieved by acetylation and/or hydroxylation of the PAMAM terminal amine groups, although the present invention is not limited to acetylation or hydroxylation.

Preferred embodiments of the present invention provide compositions comprising a nanodevices conjugated to one or more functional groups, the functional groups including, biological monitoring components, targeting components, and components to identify the specific signature of cellular abnormalities. As such, the FRET-based apoptosis detection nanodevice is made up of individual dendrimers, each with one or more functional groups being specifically conjugated with or covalently linked to the nanodevice.

Cell Targeting Components

As described above, another component of the present invention is that the FRET-based apoptosis compositions are able to specifically target a particular cell type (e.g., tumor cell). Although an understanding of the mechanism is not necessary to practice the present invention, and the present invention is not limited to any particular mechanism of action, in some embodiments, the FRET-based apoptosis nanodevice targets a cell (e.g., a neoplastic cell) through a cell surface moiety and is taken into the cell through receptor mediated endocytosis. The expression of a number of different cell surface receptors finds use as targets for the binding and uptake of the FRET-based apoptosis nanodevice. Such receptors include, but are not limited to, EGF receptors, folate receptors, FGR receptor 2s, and the like.

Any moiety known to be located on the surface of target cells (e.g. tumor cells) finds use with the present invention. For example, an antibody directed against such a moiety targets the compositions of the present invention to cell surfaces containing the moiety. Alternatively, the targeting moiety may be a ligand directed to a receptor present on the cell surface or vice versa. In a preferred embodiment of the present invention, the targeting moiety is the folic acid receptor. In some embodiments, the targeting moiety is an RGD peptide receptor (e.g., αᵥβ₃ integrin). Similarly, vitamins also may be used to target the therapeutics (e.g., DENPs comprising a therapeutic agent) of the present invention to a particular cell. Receptors and their related ligands that find use in the context of the present invention include, but are not limited to, the folate receptor, adrenergic receptor, growth hormone receptor, luteinizing hormone receptor, estrogen receptor, epidermal growth factor receptor, fibroblast growth factor receptor, and the like. In some embodiments, for cancer (e.g., breast cancer), the cell surface may be targeted with folic acid, EGF, and FGF.

Microscopic Apoptosis Imaging

In some embodiments, the FRET-based apoptosis nanodevice of the present invention may comprise one or more additional imaging agents. For example, in some embodiments, the imaging agent is a fluorescing agent (e.g., fluorescein isothiocyanate). In some embodiments of the present invention, imaging is based on the passive or active observation of local differences in density of selected physical properties of cells undergoing apoptosis. These differences may be due to a different shape (e.g., mass density detected by atomic force microscopy), altered composition (e.g. radiopaques detected by X-ray), distinct light emission (e.g., fluorochromes detected by spectrophotometry), different diffraction (e.g., electron-beam detected by TEM), contrasted absorption (e.g., light detected by optical methods), or special radiation emission (e.g., isotope methods), etc. Thus, quality and sensitivity of imaging depend on the property observed and on the technique used. The imaging techniques for cancerous cells provide sufficient levels of sensitivity to observe small, local concentrations of selected cells. The earliest identification of cancer signatures requires high selectivity (i.e., highly specific recognition provided by appropriate targeting) and the highest possible sensitivity.

Static structural microscopic imaging of cancerous cells and tissues has traditionally been performed outside of the patient. Classical histology of tissue biopsies provides an illustrative example, and has proven a powerful adjunct to cancer diagnosis and treatment. After removal, a specimen is sliced thin (e.g., less than 40 microns), stained, fixed, and examined by a pathologist. If images are obtained, they are most often 2-D transmission bright-field projection images. Specialized dyes are employed to provide selective contrast, which is almost absent from the unstained tissue, and to also provide for the identification of aberrant cellular constituents. Quantifying sub-cellular structural features by using computer-assisted analysis, such as in nuclear ploidy determination, is often confounded by the loss of histologic context owing to the thinness of the specimen and the overall lack of 3-D information. Despite the limitations of the static imaging approach, it has been invaluable to allow for the identification of neoplasia in biopsied tissue. Furthermore, its use is often the crucial factor in the decision to perform invasive and risky combinations of chemotherapy, surgical procedures, and radiation treatments, which are often accompanied by severe collateral tissue damage, complications, and even patient death.

The FRET-based apoptosis nanodevices of the present invention allow functional microscopic imaging of tumors and provide improved methods for imaging. The methods find use in vivo, in vitro, and ex vivo. For example, in one embodiment of the present invention, FRET-based apoptosis nanodevices of the present invention are designed to emit fluorescent signals. In some embodiments of the present invention, sensing fluorescent biosensors in a microscope involves the use of tunable excitation and emission filters and multiwavelength sources (Farkas et al., SPEI 2678:200 (1997)). In embodiments where the imaging agents are present in deeper tissue, longer wavelengths in the Near-infrared (NIR) are used (See e.g., Lester et al., Cell Mol. Biol. 44:29 (1998)). Dendrimeric biosensing in the Near-IR has been demonstrated with dendrimeric biosensing antenna-like architectures (Shortreed et al., J. Phys. Chem., 101:6318 (1997)). Biosensors that find use with the present invention include, but are not limited to, fluorescent dyes and molecular beacons.

Evaluation of Anti-Tumor Efficacy and Toxicity Using the FRET-Based Apoptosis Nanodevice

In some embodiments, the FRET-based apoptosis detectors of the present invention are used in monitoring during cancer therapy. However, the systems and compositions of the present invention find use in the monitoring of a variety of disease states or other physiological conditions, and the present invention is not limited to use with any particular disease state or condition. Other disease states that find particular use with the present invention include, but are not limited to, cardiovascular disease, viral disease, inflammatory disease, and other proliferative disorders.

The present invention provides the opportunity to monitor therapeutic success following delivery of a therapeutic (e.g., methotrexate and/or cisplatin and/or Taxol) to a subject. For example, measuring the ability of these drugs to induce apoptosis in vitro is a marker for in vivo efficacy (Gibb, Gynecologic Oncology 65:13 (1997)). Therefore, the effectiveness of a therapy can be monitored by techniques of the present invention that monitor the induction of apoptosis. Importantly, these diagnostics are useful within a wide range of tumor types including, but not limited to, breast cancer and colon cancer.

The anti-tumor effects of various therapeutic agents on cancer cell lines and primary cell cultures may be evaluated using the FRET-based apoptosis nanodevices of the present invention. For example, in preferred embodiments, assays are conducted, in vitro, using established tumor cell line models or primary culture cells, or alternatively, assays can be conducted in vivo using animal models.

Biological Monitoring of Apoptosis

The biological monitoring or sensing component of the FRET-based apoptosis nanodevices of the present invention is one which that can monitor the particular response in the tumor cell induced by an agent (e.g., a therapeutic agent provided by the therapeutic component). While the present invention is not limited to any particular monitoring system, the invention is illustrated by methods and compositions for monitoring cancer treatments. In preferred embodiments of the present invention, the agent induces apoptosis in cells and monitoring involves the detection of apoptosis. In particular embodiments, the monitoring component is an agent that fluoresces at a particular wavelength when apoptosis occurs. For example, in a preferred embodiment, caspase activity activates green fluorescence in the monitoring component. Apoptotic cancer cells, which have turned red as a result of being targeted by a particular signature with a red label, turn orange while residual cancer cells remain red. Normal cells induced to undergo apoptosis (e.g., through collateral damage), if present, will fluoresce green.

In these embodiments, fluorescent groups such as fluorescein are employed in the monitoring component. Fluorescein is easily attached to the dendrimer surface via the isothiocyanate derivatives, available from Molecular Probes, Inc. (Carlsbad, Calif.). This allows the nanodevices to be imaged with the cells via confocal microscopy. Sensing of the effectiveness of the FRET-based apoptosis nanodevices is preferably achieved by using fluorogenic peptide enzyme substrates. For example, apoptosis caused by the therapeutic agents results in the production of the peptidase caspase-1 (ICE). Calbiochem (San Diego, Calif.) sells a number of peptide substrates for this enzyme that release a fluorescent moiety. Thus the appearance of green fluorescence in the target cells produced using these methods provides a clear indication that apoptosis has begun.

Additional fluorescent dyes that find use with the present invention include, but are not limited to, acridine orange, reported as sensitive to DNA changes in apoptotic cells (Abrams et al., Development 117:29 (1993)) and cis-parinaric acid, sensitive to the lipid peroxidation that accompanies apoptosis (Hockenbery et al., Cell 75:241 (1993)). It should be noted that the peptide and the fluorescent dyes are merely exemplary. It is contemplated that any peptide that effectively acts as a substrate for a caspase produced as a result of apoptosis finds use with the present invention.

Quantifying the Induction of Apoptosis of Human Tumor Cells In Vitro

In an exemplary embodiment of the present invention, the FRET-based apoptosis nanodevices of the present invention are used to assay apoptosis of human tumor cells in vitro. Testing for apoptosis in the cells determines the efficacy of the therapeutic agent. Multiple aspects of apoptosis can and should be measured. These aspects include those described above, as well as aspects including, but not limited to, measurement of phosphatidylserine (PS) translocation from the inner to outer surface of plasma membrane, measurement of DNA fragmentation, detection of apoptosis related proteins, and measurement of Caspase-3 activity.

In Vitro Toxicology

In some embodiments of the present invention, toxicity testing is performed. Toxicological information may be derived from numerous sources including, but not limited to, historical databases, in vitro testing, and in vivo animal studies. In vitro toxicological methods have gained popularity in recent years due to increasing desires for alternatives to animal experimentation and an increased perception to the potential ethical, commercial, and scientific value. In vitro toxicity testing systems have numerous advantages including improved efficiency, reduced cost, and reduced variability between experiments. These systems also reduce animal usage, eliminate confounding systemic effects (e.g., immunity), and control environmental conditions.

Although any in vitro testing system may be used with the present invention, the most common approach utilized for in vitro examination is the use of cultured cell models. These systems include freshly isolated cells, primary cells, or transformed cell cultures. Cell culture as the primary means of studying in vitro toxicology is advantageous due to rapid screening of multiple cultures, usefulness in identifying and assessing toxic effects at the cellular, subcellular, or molecular level. In vitro cell culture methods commonly indicate basic cellular toxicity through measurement of membrane integrity, metabolic activities, and subcellular perturbations. Commonly used indicators for membrane integrity include cell viability (cell count), clonal expansion tests, trypan blue exclusion, intracellular enzyme release (e.g. lactate dehydrogenase), membrane permeability of small ions (K¹, Ca²⁺), and intracellular Ala accumulation of small molecules (e.g., ⁵¹Cr, succinate). Subcellular perturbations include monitoring mitochondrial enzyme activity levels via, for example, the MTT test, determining cellular adenine triphosphate (ATP) levels, neutral red uptake into lysosomes, and quantification of total protein synthesis. Metabolic activity indicators include glutathione content, lipid peroxidation, and lactate/pyruvate ratio.

In Vivo Imaging of Apoptosis

In some embodiments of the present invention, in vivo imaging is accomplished using functional imaging techniques. Functional imaging is a complementary and potentially more powerful technique as compared to static structural imaging. Functional imaging is best known for its application at the macroscopic scale, with examples including functional Magnetic Resonance Imaging (fMRI) and Positron Emission Tomography (PET). However, functional microscopic imaging may also be conducted and find use in in vivo and ex vivo analysis of living tissue. Functional microscopic imaging is an efficient combination of 3-D imaging, 3-D spatial multispectral volumetric assignment, and temporal sampling: in short a type of 3-D spectral microscopic movie loop. Interestingly, cells and tissues auto fluoresce. When excited by several wavelengths, providing much of the basic 3-D structure needed to characterize several cellular components (e.g., the nucleus) without specific labeling. Oblique light illumination is also useful to collect structural information and is used routinely. As opposed to structural spectral microimaging, functional spectral microimaging may be used with biosensors, which act to localize physiologic signals within the cell or tissue. For example, in some embodiments of the present invention, biosensor-comprising FRET-based apoptosis nanodevices of the present invention are used to image upregulated receptor families such as the folate or EGF classes. In such embodiments, functional biosensing therefore involves the detection of physiological abnormalities relevant to carcinogenesis or malignancy, even at early stages. In other embodiments, a two-photon optical fiber device may be inserted through 1 27-gauge needle to quantify the fluorescence of a targeted nanodevice in live organism tumors. (Thomas T P, Myaing M T, Ye J Y, Candido K, Kotylar A, Beals J, Cao P, Keszler B, Norris T B, Baker J R. Detection and analysis of tumor fluorescence using a two-photon optical fiber probe. Biophys. J. 2004, 86, 3959-3965.) A number of physiological conditions may be imaged using the compositions and methods of the present invention including, but not limited to, detection of nanoscopic dendrimeric biosensors for pH, oxygen concentration, Ca²⁺ concentration, and other physiologically relevant analytes.

Once the apoptosis nanodevice has attached to (or been internalized into) tumor cells, one or more modules of the apoptosis nanodevice (e.g., a metal nanoparticle encapsulated by the dendrimer, and/or, an imaging agent conjugated to the dendrimer) may serve to image its location. Dendrimers have been employed as biomedical imaging agents, perhaps most notably for magnetic resonance imaging (MRI) contrast enhancement agents (See e.g., Wiener et al., Mag. Reson. Med. 31:1 (1994); an example using PAMAM dendrimers). These agents are typically constructed by conjugating chelated paramagnetic ions, such as Gd(III)-diethylenetriaminepentaacetic acid (Gd(III)-DTPA), to water-soluble dendrimers. Other paramagnetic ions that may be useful in this context of the invention include, but are not limited to, gadolinium, manganese, copper, chromium, iron, cobalt, erbium, nickel, europium, technetium, indium, samarium, dysprosium, ruthenium, ytterbium, yttrium, and holmium ions and combinations thereof. In some embodiments of the present invention, the dendrimer is also conjugated to a targeting group, such as epidermal growth factor (EGF), to make the conjugate specifically bind to the desired cell type (e.g., in the case of EGF, EGFR-expressing tumor cells). In a preferred embodiment of the present invention, DTPA is attached to dendrimers via the isothiocyanate of DTPA as described by Wiener (Wiener et al., Mag. Reson. Med. 31:1 (1994)).

MRI agents are particularly effective due to the polyvalency, size and architecture of apoptosis nanodevices (e.g., comprising both dendrimers conjugated to one or more functional groups and an encapsulated metal nanoparticle), which results in molecules with large proton relaxation enhancements, high molecular relaxivity, and a high effective concentration of paramagnetic ions at the target site. Dendrimeric gadolinium contrast agents have even been used to differentiate between benign and malignant breast tumors using dynamic MRI, based on how the vasculature for the latter type of tumor images more densely (Adam et al., Invest. Rad. 31:26 (1996)). Thus, MRI provides a particularly useful imaging system of the present invention.

### EXPERIMENTAL

In the experimental disclosure which follows, the following abbreviations apply: g (grams); 1 or L (liters); μg (micrograms); μl (microliters); μm (micrometers); μM (micromolar); μmol (micromoles); mg (milligrams); ml (milliliters); mm (millimeters); mM (millimolar); mmol (millimoles); M (molar); mol (moles); ng (nanograms); nm (nanometers); nmol (nanomoles); N (normal); and pmol (picomoles).

### Experimental Procedures

Syntheses

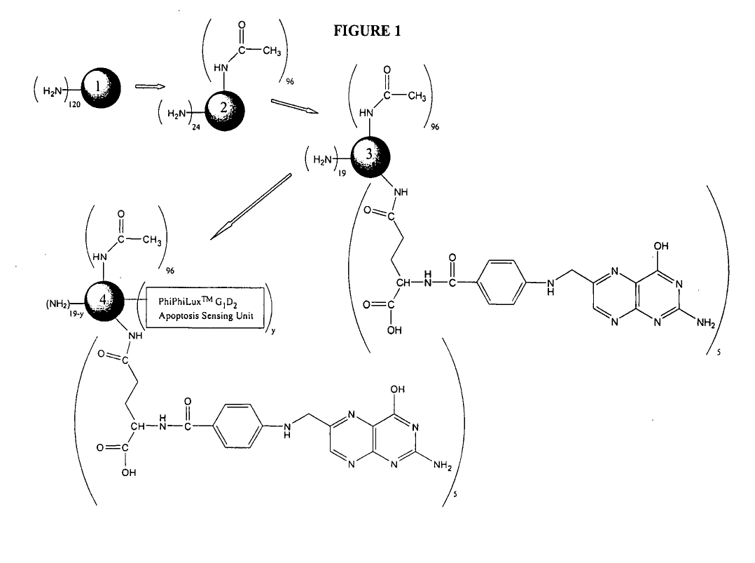

An exemplary synthetic scheme for production of dendritic devices is provided in FIG. 1.

1. G5 carrier: The PAMAM G5 dendrimer (called gold standard, DRS-526-26) was synthesized and characterized at the Michigan Nanotechnology Institute for Medicine and Biological Sciences MNIMBS), University of Michigan. The synthesized dendrimer was analyzed by using NMR, HPLC, GPC and potentiometric titration. The molecular weight was found to be 26,380 g/mol by GPC and the average number of primary amino groups was determined by potentiometric titration to be 120. These two analytical data are important to design chemical reaction precisely.

2. G5-Ac(96): 0.2071 g (7.85×10⁻⁶ mol) of G5 PAMAM dendrimer (MW=26,380 g/mol by GPC, number of primary amines=120 by potentiometric titration) in 16 ml of abs. MeOH was allowed to react with 59.3 μl (6.28×10⁻⁴ mol) of acetic anhydride in the presence of 109.4 μL (7.85×10⁻⁴ mol, 25% molar excess) triethylamine (reaction time 14 hours). After intensive dialysis in DI water and lyophilization, 223.0 mg (93.4%) of G5-Ac(96) product was yielded. The average number of acetyl groups (96) was determined based on ¹H NMR calibration. (Majoros, I. J., Keszler, B., Woehler, S., Bull, T., and Baker, Jr. J. R. (2003). Acetylation of Poly(amidoamine) Dendrimers. Macromolec. 36, 5526-5529.)

3. G5-Ac(96)-FA: FA was attached to G5-Ac(96) in two consecutive reactions. 0.0028 g (6.343×10⁻⁶ mol) FA was allowed to react with a 14-fold excess of EDC 0.01707 g (8.906×10⁻⁵ mol) in a solvent mixture of 3 ml of DMF and 1 ml of DMSO at r.t. (reaction time 1 h), and then this FA-active ester solution was added drop wise to an aqueous solution of the partially acetylated product G5-Ac(96) (0.0126 g, 4.143×10⁻⁷ mol) in 12 mL of water (reaction time 3 days). After dialysis in DI water, repeated membrane filtration (using PBS and DI water) and lyophilization, the product weight was 0.01209 g (95.45%). The number of FA molecules (to be 5) was determined by proton NMR spectroscopy. As an additional characterization, no free FA was observed by a HPLC or by agarose gel.

4. G5-Ac(96)-FA-PhiPhiLux™ G₁D₂: PhiPhiLux™ G₁D₂ was attached to G5-Ac(96)-FA mono-functional dendrimer conjugate in two consecutive reactions. 0.0013 g (6.685×10⁻⁷ mol) PhiPhiLux™ G₁D₂ (MW=1944.73 g/mol) was allowed to react with a 14-fold excess of EDC 0.0018 g (9.389×10⁻⁶ mol) in a solvent mixture of 3 mL of DMF and 1 mL of DMSO at room temperature (reaction time 1 h), and then this PhiPhiLux™ G₁D₂-active ester solution was added drop wise to an aqueous solution of the partially acetylated mono-functional dendrimer conjugate G5-Ac(96)-FA (0.0023 g, 7.05×10⁻⁸ mol) in 12 mL of water (reaction time 2 days). After repeated membrane filtration (using PBS and DI water), and lyophilization, the product weight was 0.0033 g. This bi-functional dendrimer conjugate was used for biological testing.

Materials:

The G5 PAMAM dendrimer was synthesized and characterized at the Michigan Nanotechnology Institute for Medicine and Biological Sciences, University of Michigan. Methanol (MeOH, HPLC grade), acetic anhydride (99%), triethylamine (99.5%), DMSO (99.9%), DMF (99.8%), 1-[3-(Dimethylamino)-propyl]-3-ethylcarbodiimide HCl (EDC, 98%), citric acid (99.5%), sodium azide (99.99%), D₂O, NaCl, and volumetric solutions (0.1M HCl and 0.1M NaOH) for potentiometric titration were purchased from Aldrich Co. and used as received. The FA and staurosporine were purchased from Sigma (St. Louis, Mo.). Spectra/Por®, dialysis membrane (MWCO 3,500), Millipor Centricon ultrafiltration membrane YM-10 and phosphate buffer saline (PBS, pH 7.4) were purchased from Fisher. PhiPhiLux™ G₁D₂ was purchased from Calbiochem (San Diego, Calif.). The Jurkat E6 and KB cell lines were purchased from American Type Cell Collection (ATCC, Manassas, Va., USA) and grown on RPMI medium supplemented with penicillin (100 units/mL), streptomycin (100 μG/mL), 50 mM L-glutamine, and 10% heat-inactivated FBS, as monolayer at 37° C. and 5% CO₂. The UMSCC-38 head and neck squamous carcinoma cell line (1) was kindly provided by Dr. J. Mulè (University of Michigan).

Potentiometric Titration:

Titration was carried out manually using a Mettler Toledo MP230 pH Meter and MicroComb pH electrode at room temperature, 23±1° C. A 10 mL solution of 0.1 M NaCl was added to precisely weighed 118.4 mg of G5 PAMAM dendrimer to shield amine group interactions. Titration was performed with 0.1037 N HCl, and 0.1033 N NaOH was used for back titration. The numbers of primary and tertiary amines were determined from back titration data.

Gel Permeation Chromatography:

GPC experiments were performed on an Alliance Waters 2690 Separation Module equipped with 2487 Dual Wavelength UV Absorbance Detector (Waters Corporation), a Wyatt Dawn® DSP Laser Photometer, an Optilab DSP Interferometric Refractometer (Wyatt Technology Corporation), and with TosoHaas TSK-Gel® Guard PHW 06762 (75×7.5 mm, 12 μm), G 2000 PW 05761 (300×7.5 mm, 10 μm), G 3000 PW 05762 (300×7.5 mm, 10 μm), and G 4000 PW (300×7.5 mm, 17 μm) columns. Column temperature was maintained at 25±0.1° C. by a Waters Temperature Control Module. The isocratic mobile phase was 0.1 M citric acid and 0.025 wt % sodium azide, pH 2.74, at a flow rate of 1 mL/min. Sample concentration was 10 mg/5 mL with an injection volume of 100 μL. The molecular weight and molecular weight distribution of the PAMAM dendrimer and its conjugates were determined using Astra 4.7 software (Wyatt Technology Corporation).

Nuclear Magnetic Resonance Spectroscopy:

¹H and ¹³C NMR spectra were taken in D₂O and were used to provide integration values for structural analysis by means of a Bruker AVANCE DRX 500 instrument.

UV Spectrophotometry:

UV spectra were recorded using a Perkin Elmer UV/VIS Spectrometer Lambda 20 and Lambda 20 software, in PBS.

Reverse Phase High Performance Liquid Chromatography:

A reverse phase ion-pairing high performance liquid chromatography (RP-HPLC) system consisted of a System GOLD™ 126 solvent module, a Model 507 auto sampler equipped with a 100 μL loop, and a Model 166 UV detector (Beckman Coulter, Fullerton, Calif.). A Phenomenex (Torrance, Calif.) Jupiter C5 silica based HPLC column (250×4.6 mm, 300 Å) was used for the separation of analytes. Two Phenomenex safety guards were also installed upstream of the HPLC column. The mobile phase for elution of PAMAM dendrimers was a linear gradient beginning with 90:10 water/ acetonitrile (ACN) at a flow rate of 1 mL/min, reaching 50:50 after 30 minutes. Trifluoroacetic acid (TFA) at 0.14 wt % concentration in water as well as in ACN was used as counter-ion to make the dendrimer-conjugate surfaces hydrophobic. The conjugates were dissolved in the mobile phase (90:10 water/ACN). The injection volume in each case was 50 μL with a sample concentration of approximately 1 mg/mL, and the detection of eluted samples was performed at 210, 242, or 280 nm. The analysis was performed using Beckman's System GOLD™ Nouveau software. Characterization of all intermediates has been performed through the use of UV, HPLC, NMR, and GPC.

Cell Culture and Treatment:

The KB cell line (ATCC, Manassas, Va., USA) is a human epidermoid carcinoma that over-expresses folate receptors, especially when grown in low folic acid medium. (Antony, A. C.; Kane, M. A.; Portillo, R. M.; Elwood, P. C.; and Kolhouse, J. F. (1985). Studies of the role of a particulate folate-binding protein in the uptake of 5-methyltetrahydrofolate by cultured human KB cells. J. Biol. Chem. 260, 14911-14917.) The KB cells were grown continuously as a monolayer at 37° C. and 5% CO₂ in folic acid-deficient RPMI 1640 medium. This medium was supplemented with penicillin (100 units/mL), streptomycin (100 μL/mL), and 10% heat-inactivated FBS, yielding a final folic acid concentration approximately that of normal human serum. Approximately 2×10⁴ cells per well were seeded the day before experiments in 12-well plates, either with complete medium (KB folate receptor down-regulated cells) or folic acid-deficient medium (KB folate receptor up-regulated cells). An hour before each experiment, the cells were washed with their respective media, then 500 μL of either the complete medium or folic acid-deficient medium were put in each well. An hour later, the cells were treated with either G5-Ac(96)-FA-PhiPhiLux™ G₁D₂, the control solution, or free PhiPhiLux™ G₁D₂. After one-hour of treatment, the cells were washed with PBS and fresh medium was added to each well. After incubation for an additional 72 hours, the cells were harvested and washed with PBS containing 0.1% bovine serum albumin (BSA) before analysis by flow cytometry. In some experiments, the KB cells were treated for 72 hours and then analyzed.

Flow Cytometric Analysis:

To estimate the cell death, the KB cells were incubated with propidium iodide (1.25 μg/mL) for 5 minutes at room temperature. The dead cells are not able to exclude propidium iodide dye, and thereby dye binds to cellular nucleic acids generating red fluorescence in the cells. However, the living cells exclude propidium iodide and remain non-fluorescent. After incubation with propidium iodide, the cells were acquired on a Beckman-Coulter EPICS-XL MCL flow cytometer, and data was analyzed using Expo32 software (Beckman-Coulter, Miami, Fla.).

### EXAMPLES

### Example 1

### Dendrimer Synthesis

Partial Acetylation

The PAMAM dendrimer used in this study was uniform, monodispersed and GMP grade. The full characterization of the PAMAM dendrimer has been made through use of gel permeation chromatography (GPC), high performance liquid chromatography (HPLC), ¹H and ¹³C NMR, and potentiometric titration. Determination of molecular weight and the number of primary amino groups were fundamental in designing reactions resulting in the synthesis of a precise conjugate structure.

By possessing the ability to synthesize a stable, unique conjugate structure capable of targeted apoptosis sensor delivery and detecting apoptosis within the targeted cell(s), molecular semi-engineering allows the capability of synthesizing complex yet well-defined devices, which is a key principle of targeted apoptosis detection technology. Side reactions such as bridging, as well as production of fewer arms per generation than theoretically expected, aid in producing a structure slightly different from the theoretical representation of the G5 PAMAM dendrimer. The chemical structure of a G5 PAMAM dendrimer exhibits missing arms especially from higher generations (4 and 5). Precise characterization of the PAMAM dendrimer platform allows for the design of reaction sequences with stoichiometry suitable for synthesis of engineered complex macromolecules. The conjugated molecules enhance apoptosis detection through targeted, controlled delivery in response to enzymatic biochemical mechanisms.

Partial acetylation is the first reaction step in the synthesis of bi-functional device. Enhanced analytical technique allows for the precise determination of the number average number of tertiary and primary amino groups, which is necessary in order to determine the extent of the reactions required to partially acetylate the terminal amino groups.

Potentiometric titration was performed to determine the number average number of tertiary and primary amino groups. G5 PAMAM dendrimer, theoretically, has 126 tertiary and 128 primary amino groups. These values can be calculated through the use of standard mathematical formulas. (Esfand, R., Tomalia, D. A. (2001). Poly(amidoamine) (PAMAM) dendrimers: From biomimicry to drug delivery and biomedical application. Drug Discovery Today 6, 427-436, Tomalia, D. A., Baker, H., Dewald, J., Hall, M., Kallos, G., Martin, S., Roeck, J., Ryder, J., Smith, P. (1985). A new class of polymers: Starburst-dendritic macromolecules, Polym. J. 17, 117-132, Majoros, I. J., Mehta, C. B., and Baker, Jr. J. R. (2004). Mathematical description of dendrimer structure. J. Comp. Theo. Nanosci. 1, 193-198). Potentiometric titration revealed that there were 120 primary amino groups present on the dendrimer surface. A 10 mL solution of 0.1 M NaCl was added to precisely weighed 118.4 mg of G5 PAMAM dendrimer (batch: DSR-526-27) to shield amine group interactions. Titration was performed with 0.1037 N HCl, and 0.1033 N NaOH was used for back titration. The number average numbers of primary and tertiary amines were calculated using data from back-titration.

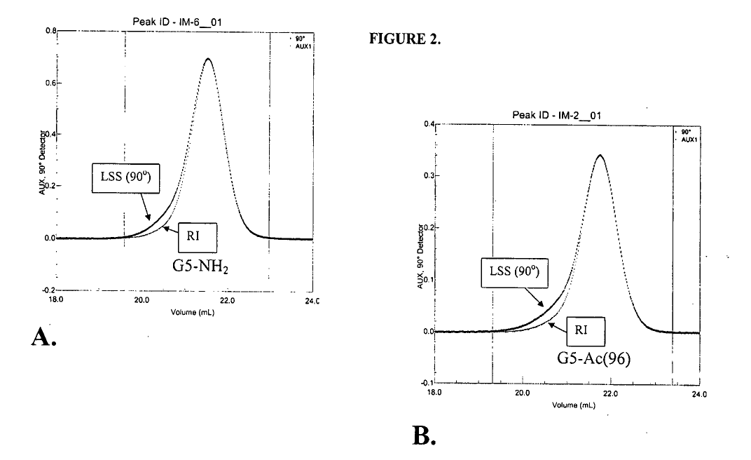

Partial acetylation is used to neutralize a fraction of the dendrimer device surface from further reaction or intermolecular interaction within the biological system, thereby preventing unwanted interactions from occurring during synthesis and during device delivery. Leaving a fraction of the primary amines nonacetylated allows for the attachment of required important molecules. The acetylation was performed in absolute MeOH with a calculated amount of acetic anhydride in the presence of triethylamine. Membrane filtration was used for purification in PBS and DI water. The average number of acetyl groups (96) was determined based on GPC and ¹H NMR calibration. (Majoros, I. J., Keszler, B., Woehler, S., Bull, T., and Baker, Jr. J. R. (2003). Acetylation of Poly(amidoamine) dendrimers. Macromolec. 36, 5526-5529). FIG. 2 shows gel permeation chromatography eluograms of the G5 dendrimer and partially acetylated G5 carrier with the RI signal and laser light scattering signal overlapping at 90°, indicating that there is no defect in the analyzed structure.

Folic Acid Conjugation

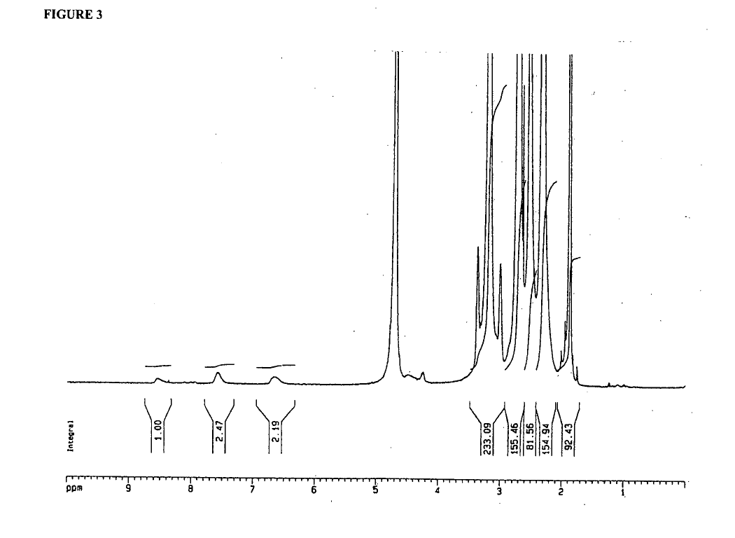

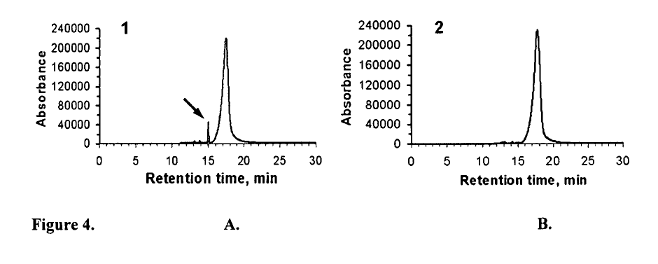

In the next reaction, folic acid (FA) was attached to the G5-Ac(96) carrier. When the γ-carboxylic group of FA is used for conjugation, FA retains a strong affinity toward its receptor, allowing the FA moiety of the conjugate to retain its ability to act as a targeting function. Additionally, the γ-carboxylic group possesses a higher reactivity during carbodiimide-mediated coupling to primary amino groups as compare to the α-carboxyl group. (Quintana, A., Raczka, E., Piehler, L., Lee, I., Myc, A., Majoros, I., Patri, A., Thomas, T., Mulé, J., and Baker, Jr. J. (2002). Design and function of a dendrimer-based therapeutic nanodevice targeted to tumor cells through the folate receptor, Pharm. Res. 19, 1310-1316). Conjugation of FA to the partially acetylated dendrimer was carried out via condensation between the γ-carboxyl group of FA and the primary amino groups of the dendrimer. The active ester of FA, formed by reaction with EDC in DMF-DMSO (3:1 solvent mixture), was added drop-wise to a solution of DI water containing G5-Ac(96) and was vigorously stirred for 3 days to allow for the FA to conjugate to the G5-Ac(96). (Quintana, A., Raczka, E., Piehler, L., Lee, I., Myc, A., Majoros, I., Patri, A., Thomas, T., Mule, J., and Baker, Jr. J. (2002). Design and function of a dendrimer-based therapeutic nanodevice targeted to tumor cells through the folate receptor, Pharm. Res. 19, 1310-1316). NMR was also used to confirm the number of FA molecules attached to the dendrimer (FIG. 3). If free FA were present in the sample, sharp peaks would appear in the spectrum (at the broad aromatic peaks). The broadening of the aromatic proton peaks in the G5-Ac(96)-FA spectrum indicates the presence of a covalent bond between the FA and the dendrimer. Based on the integration values of the methyl protons in the acetamide groups (1.84 ppm), and the aromatic protons in the FA (6.64, 7.55 and 8.52 ppm), the number of attached FA molecules was calculated to be 4.9. The number of FA molecules (5.3), was determined by UV spectroscopy, utilizing the concentration calibration curve of free FA. For quality control purpose HPLC has been used. The HPLC eluogram (FIG. 4) of the G5-Ac(96)-FA(5) conjugate clearly indicates presence of free FA before membrane filtration purification (1) in comparison with eluogram recorded after purification (2).

PhiPhiLux™ G₁D₂ Conjugation

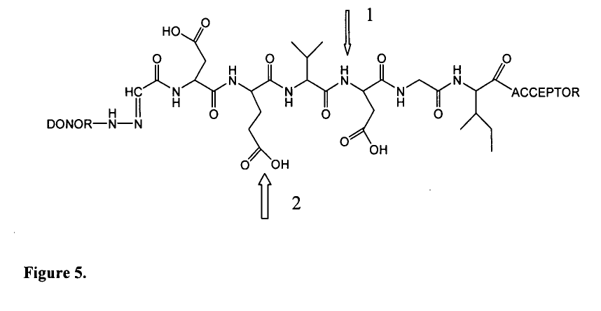

PhiPhiLux™ G₁D₂ (FIG. 5) was attached to G5-Ac(96)-FA mono-functional dendrimer conjugate in two consecutive reactions. PhiPhiLux™ G₁D₂ was allowed to react with a 14-fold excess of EDC in a solvent mixture of DMF:DMSO (3:1) at room temperature for 1 h, and then the PhiPhiLux™ G₁D₂-active ester solution was added dropwise to an aqueous solution of the partially acetylated mono-functional dendrimer conjugate G5-Ac(96)-FA in DI water at room temperature for 2 days. After repeated membrane filtration (using PBS and DI water), and lyophilization, the final amount of the product was 3.3 mg. This bi-functional dendrimer conjugate was used for biological testing.

### Example 2

### Apoptosis Detection in Jurkat Cells

The potential of the Caspase-3 Intracellular Activity Assay Kit I (PhiPhiLux™ G₁D₂), Cat. No. 235430 (Calbiochem) was tested for the use in a novel bi-functional G5dendrimer conjugate to specifically detect apoptosis. The FRET-based apoptosis detector (PhiPhiLux™ G₁D₂) was examined to discriminate between control cells and cells which undergone apoptosis. FRET detection measures non-covalent bonding events in biological and macromolecular systems. Due to the presence of caspase-3, which cleaves certain cellular substrates during apoptosis, and the effects of caspase-3 on fluorescence resonance energy transfer, FRET detection can determine whether apoptosis has occurred. In order for the FRET effect to occur, it is necessary for the fluorescence emission band of the donor fluorophore molecule to overlap with the excitation band of the acceptor molecule within 20-80 Å of the donor. (Stauffer, S. R., and. Hartwig, J. F. (2003). Fluorescence resonance energy transfer (FRET) as a high-throughput assay for coupling reactions. Arylation of amines as a case study. J. Am. Chem. Soc. 125, 6977-8985). Some FRET reagents yield a relatively high level of background fluorescence, and therefore the difference between nonspecific and specific staining is minimal. The transfer of energy due to the FRET effect that can be detected by the temporal increase in fluorescent intensity by the acceptor is called “acceptor in-growth”. (Stauffer, S. R., and. Hartwig, J. F. (2003). Fluorescence Resonance Energy Transfer (FRET) as a High-Throughput Assay for Coupling Reactions. Arylation of Amines as a Case Study. J. Am. Chem. Soc. 125, 6977-8985). The presence of caspase-3, which is only active during apoptosis, is detected by the elimination of the FRET effect. Resulting cleavage of the peptide by caspase-3, between valine and aspartic acid in the recognition sequence D-E-V-D, results in the elimination of the FRET effect because the donor and acceptor fluorophores are no longer joined. Flow cytometry is used to quantify the amount of fluorescence present. By observing intensity shift between the emissions of the donor and acceptor fluorophores, it is possible to determine the change in the FRET effect as a function of the cleavage of the linker by the enzyme caspase-3. (Luo, K. Q., Yu, V. C., Pu, Y., and Chang, D. C. (2001). Application of the fluorescence resonance energy transfer method for studying the dynamics of caspase-3 activation during UV-induced apoptosis in Living HeLa Cells. Biochem. Biophys Res. Comm. 283, 1054-1060).

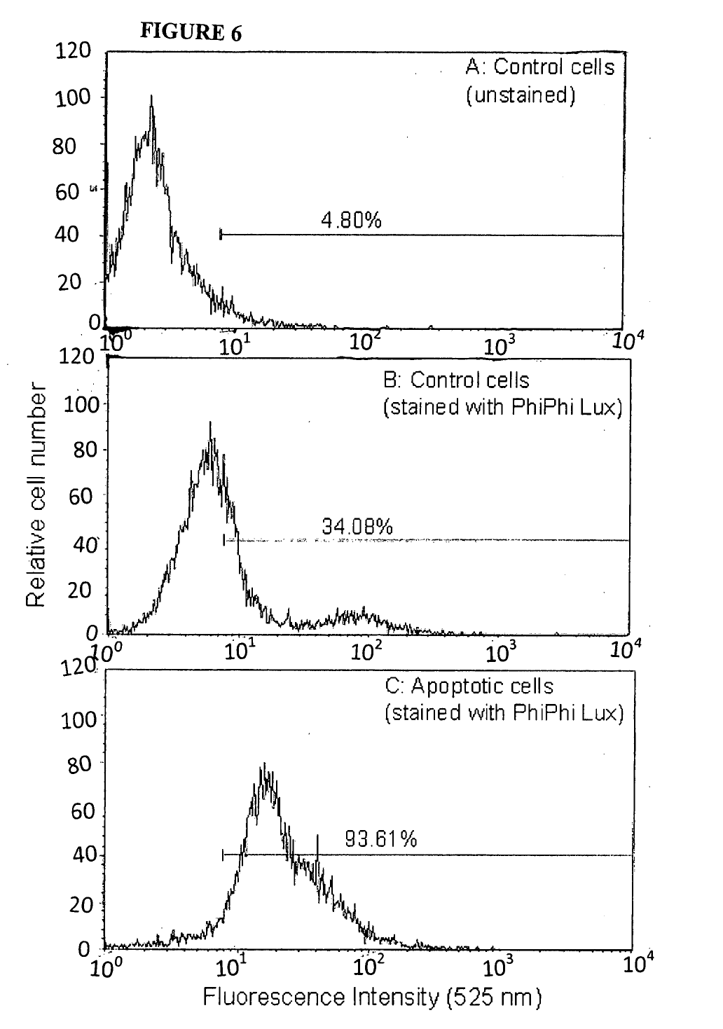

As shown in FIG. 6, control Jurkat cells non-specifically stained with PhiPhiLux™ G₁D₂ yielded approximately 34% positive cells (FIG. 6B) as compared to unstained control Jurkat cells (FIG. 6A). Apoptotic Jurkat cells showed a further increase in fluorescence intensity yielding approximately 93% positive cells (FIG. 6C). Although the background fluorescence was present, significant differences in fluorescence between control and apoptotic cells were observed.

### Example 3

### Apoptosis Detection in KB and UMSCC-38 Cells

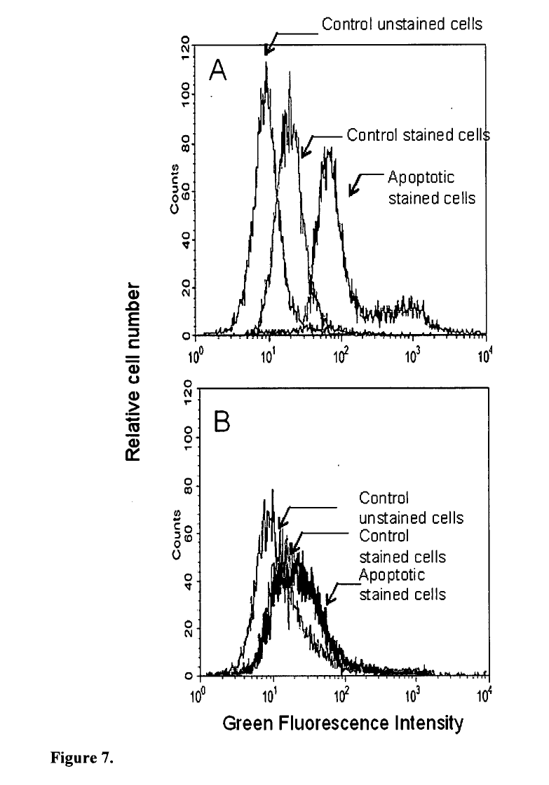

The newly synthesized G5-Ac(96)-FA-PhiPhiLux™ G₁D₂ nanodevice was examined for its functionality in KB (folate receptor positive) and UMSCC-38 (folate receptor negative) cells. KB and UMSCC-38 cells were incubated with the nanodevice for 30 min prior to inducing apoptosis with Staurosporine. Three and a half hours later the cells were trypsinized, washed, and analyzed on flow cytometry to measure green fluorescence. As shown on FIG. 7, control KB cells showed minimal non-specific increase in fluorescence intensity as compared to control unstained cells. However, the apoptotic KB cells increased fluorescence intensity to a much greater degree, and were easily distinguished from nonspecifically stained control cells (FIG. 7A). To the contrary, UMSCC-38 cells shown to be apoptotic did not show any increase in fluorescence intensity over the background fluorescence of stained control cells (FIG. 7B) indicating that the nanodevice of the present invention was not internalized. These results indicate that KB cells actively internalized the nanodevice through the folate receptor during the first 30 minutes of incubation, and after induction of apoptosis the active caspase-3 cleaved the bond between donor and acceptor on PhiPhiLux™ G₁D₂ conjugated to the dendrimer, thereby increasing the fluorescence intensity in the aptoptotic KB cells. Accordingly, the conjugation to the polymer prevented internalization into receptor-negative cells.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

We claim:

1. A composition comprising a FRET-based substrate, a cell targeting moiety and a dendrimer.

2. The composition of claim 1, wherein said FRET-based substrate is PhiPhiLux™ G₁D₂.

3. The composition of claim 1, wherein said targeting moiety is folic acid.

4. The composition of claim 1, wherein said dendrimer is PAMAM G5.

5. The composition of claim 1, wherein said FRET-based substrate is PhiPhiLux™ G₁D₂, said targeting moiety is folic acid, and wherein said dendrimer is PAMAM G5.

6. A method to detect apoptosis, comprising:
a. providing:
i. a cell;
ii. a nanodevice, comprising:
a. a FRET-based substrate;
b. a cell-targeting moiety; and
c. a dendrimer, wherein said FRET-based substrate, said cell targeting moiety and said dendrimer comprise a stable conjugate; and
b. contacting said cell with said nanodevice; and
c. detecting a change in the level of an intracellular fluorescent signal indicating the presence or absence of apoptosis of said cell.

7. The method of claim 6, wherein said apoptosis is caspase-3 mediated apoptosis.

8. The method of claim 6, wherein said FRET-based substrate is PhiPhiLux™ G₁D₂.

9. The method of claim 6, wherein said targeting moiety is folic acid.

10. The method of claim 6, wherein said dendrimer is PAMAM G5.

11. The method of claim 6, wherein said cell is folate receptor α positive.

12. The method of claim 6, wherein said cell is a neoplastic cell.

13. The method of claim 6, wherein said detection is by flow cytometry.

14. The method of claim 6, wherein said detection is in vitro.

15. The method of claim 6, wherein said detection is in vivo.

16. A method of synthesizing a FRET-based apoptosis detection composition, comprising:
a) partially acetylating a dendrimer:
b) conjugating said partially acetylated dendrimer with folic acid via condensation;
c) reacting a FRET-based substrate with an excess of EDC in a mixture of DMF:DMSO; and
d) conjugating said FRET-based substrate to said partially acetylated dendrimer-folic acid conjugate.

17. The composition of claim 16, wherein said FRET-based substrate is PhiPhiLux™ G₁D₂.

18. The composition of claim 16, wherein said targeting moiety is folic acid.

19. The composition of claim 16, wherein said dendrimer is PAMAM G5.

20. A kit, comprising the composition of claim 1.
